(19) United States
(12) Patent Application Publication
(10) Pub. No.: US 2023/0000565 A1
(43) Pub. Date: Jan. 5, 2023

(54) SYSTEMS AND METHODS FOR AUTONOMOUS SUTURING

(71) Applicant: Activ Surgical, Inc., Boston, MA (US)

(72) Inventors: Michael C. Pickett, Boston, MA (US); Tina P. Chen, Boston, MA (US); Hossein Dehghani, Boston, MA (US); Vasiliy E. Buharin, Boston, MA (US); Emanuel DeMaio, Boston, MA (US); Tony Chen, Boston, MA (US); John Oberlin, Boston, MA (US); Liam O'Shea, Boston, MA (US); Thomas Calef, Boston, MA (US)

(21) Appl. No.: 17/811,942

(22) Filed: Jul. 12, 2022

Related U.S. Application Data
(63) Continuation of application No. PCT/US2021/013309, filed on Jan. 13, 2021.
(60) Provisional application No. 62/960,908, filed on Jan. 14, 2020.
(60) Provisional application No. 62/962,850, filed on Jan. 17, 2020.

(51) Int. Cl. A61B 34/20; A61B 34/32

(52) U.S. Cl. CPC A61B 34/20; A61B 34/32; A61B 2034/2065; A61B 2034/2048

(57) ABSTRACT

The present disclosure provides a system for enabling autonomous or semi-autonomous surgical operations. The system comprises: one or more processors that are individually or collectively configured to: process an image data stream comprising one or more images of a surgical site; fit a parametric model to a tissue surface identified in the one or more images; determine a direction for aligning a tool based in part on the parametric model; determine an optimal path for automatically moving the tool to perform a surgical procedure at the surgical site; and generate one or more control signals for controlling i) a movement of the tool based on the optimal path and ii) a tension force applied to the tissue by the tool during the surgical procedure.

### CROSS-REFERENCE

This application is a continuation application of International Patent Application No. PCT/US2021/013309, filed Jan. 13, 2021, which claims priority to U.S. Provisional Application No. 62/960,908, filed Jan. 14, 2020, and U.S. Provisional Application No. 62/962,850, filed Jan. 17, 2020, each of which is incorporated herein by reference in its entirety for all purposes.

### BACKGROUND

Robot surgery devices have been used to assist surgeons or human tele-operators during medical or surgical procedures. However, such robotic devices or systems may still rely on human operators to control the robotic movement or operations of the system. Autonomous robotic surgery has been challenging due to technological limitations such as lack of vision system that is capable of distinguishing and tracking target tissues in dynamic surgical environments. In particular, surgical operations involving soft tissues can be more challenging due to the unpredictable, elastic, and plastic changes in soft tissue. Unlike rigid tissue surgery, autonomous decisions and execution of surgical tasks in soft tissues are required to constantly adjust to unpredictable changes such as non-rigid deformation of the tissue as a result of cutting, suturing, or cauterizing.

### SUMMARY

The present disclosure provides systems and methods that are capable of performing autonomous robotic surgeries. The systems and methods disclosed herein may automate surgical procedures without or with little human intervention. Further, the systems and methods disclosed herein may be capable of performing autonomous surgical procedures on soft tissues. In some situations, the provided autonomous robotic system may be utilized in a minimal access surgery (also known as minimally invasive surgery) which minimizes trauma to soft tissue, reduces post-operative pain, promotes earlier mobilization, shortens hospital stays, and speeds rehabilitation. The autonomous robotic system of the present disclosure may be provided with improved real-time location tracking capability and/or customized algorithms to account for the dynamic changes in the minimally invasive surgery.

In an aspect, a system is provided for enabling autonomous or semi-autonomous surgical operations. The system comprises: one or more processors that are individually or collectively configured to: process an image data stream comprising one or more images of a surgical site; fit a parametric model to a tissue surface identified in the one or more images; determine a direction for aligning a tool based in part on the parametric model; determine an optimal path for automatically moving the tool to perform a surgical procedure at the surgical site; and generate one or more control signals for controlling i) a movement of the tool based on the optimal path and ii) a tension force applied to the tissue by the tool during the surgical procedure.

In some cases, the image data stream may comprise one or more images captured using a time of flight sensor, an RGB-D sensor, or any other type of depth sensor. The one or more images may comprise a 2D image of the surgical scene that further comprises corresponding depth information associated with the 2D image of the surgical scene. In some cases, the one or more images can comprise two or more images that correspond to the same surgical site or view, but provide alternative data representations of the same surgical site or view. In some cases, the two or more images may comprise a 2D image of the surgical scene and a corresponding depth image.

In some embodiments, the image data stream is captured using a stereoscopic camera. In some cases, the system further comprises the stereoscopic camera, and wherein the stereoscopic camera is attachable to a joint mechanism that is configured to permit the stereoscopic camera to move in at least three degrees of freedom. In some instances, the stereoscopic camera is calibrated, and wherein the one or more processors are configured to determine a registration between the calibrated stereoscopic camera and a surgical robot to which the tool is mounted. For example, the one or more processors are configured to determine the registration by calculating a transformation between (i) a set of spatial coordinates of the stereoscopic camera and (ii) a set of spatial coordinates of the joint mechanism of the surgical robot.

In some embodiments, the one or more images do not contain an image of any portion of the tool. In some cases, the one or more processors are configured to calculate a posture and a position of the tool relative to the tissue surface based at least in part on a registration between a stereoscopic camera and a surgical robot to which the tool is attached.

In some embodiments, the direction for aligning the tool is along a normal vector of a parametric surface of the parametric model and a direction defined by the stitching pattern. In some embodiments, the path is a stitching pattern and the tool is a stitching needle. In some cases, the stitching pattern is generated based on an opening at the surgical site identified from the one or more images. In some instances, the one or more processors are configured to generate the stitching pattern by identifying a longitudinal axis of the opening and a plurality of anchoring points. For example, the one or more processors are configured to determine one or more of the plurality anchoring points based in part on a user input. In some cases, the one or more processors are configured to generate the stitching pattern based on a closure changing of the opening during a suturing procedure.

In some embodiments, the one or more processors are configured to control the tension force based on a tension measured in a thread or a usage of the thread during the surgical procedure. In some embodiments, the one or more processors are configured to control the tension force based on a tension or deformation model of a tissue underlying the tissue surface. In some cases, the one or more processors are configured to construct the tension or deformation model of the tissue based on the parametric model of the tissue surface.

In some embodiments, the one or more processors are configured to control insertion of the tool via a trocar. In some cases, the one or more processors are configured to compensate a location of the tool by identifying an offset caused by an external force applied to the tool via the trocar. In some cases, the one or more processors are configured to determine the offset by comparing a measured 3D coordinates of the tool with a predicted 3D coordinates of the tool.

In some embodiments, the one or more processors are configured to determine the optimal path based in part on a cyclic movement of one or more features on the surgical site. In some cases, the one or more processors are configured to track the cyclic movement using the image data stream.

In another aspect, a method is provided for enabling autonomous or semi-autonomous surgical operations. The method comprises: (a) capturing an image data stream comprising one or more images of a surgical site; (b) generating a parametric model for a tissue surface identified in the one or more images; (c) determining a direction for aligning a tool based in part on the parametric model; (d) generating an optimal path for automatically moving the tool to perform a surgical procedure at the surgical site; and (e) generating one or more control signals for controlling i) a movement of the tool based on the optimal path and ii) a tension force applied to the tissue by the tool during the surgical procedure.

In some embodiments, the image data stream is captured using a stereoscopic camera. In some cases, the stereoscopic camera is attachable to a joint mechanism that is configured to permit the stereoscopic camera to move in at least three degrees of freedom. In some instances, the method further comprises before preforming (a), calibrating the stereoscopic camera and determining a registration between the stereoscopic camera and a surgical robot to which the tool is mounted. For example, determining the registration comprises calculating a transformation between (i) camera set of spatial coordinates of the stereoscopic camera and (ii) a set of spatial coordinates of the joint mechanism of the surgical robot.

In some embodiments, the one or more images do not contain an image of any portion of the tool. In some cases, the method further comprises calculating a posture and position of the tool relative to the tissue surface in (c) based at least in part on a registration between a stereoscopic camera and a surgical robot to which the stereoscopic camera is attached.

In some embodiments, the direction for aligning the tool is along a normal vector of a parametric surface of the parametric model. In some embodiments, the path is a stitching pattern and the tool is a stitching needle. In some cases, the stitching pattern is generated based on an opening at the surgical site identified from the one or more images. In some cases, the stitching pattern is generated by identifying a longitudinal axis of the opening and a plurality of anchoring points. In some instances, one or more of the plurality anchoring points are determined based in part on a user input. In some cases, the stitching pattern is generated based on a closure changing of the opening during a suturing procedure.

In some embodiments, controlling the tension force in (e) is based on a tension measured in a thread or a usage of the thread during the surgical procedure. In some embodiments, the tension force is controlled based on a tension or deformation model of a tissue underlying the tissue surface. In some cases, the tension or deformation model of the tissue is constructed based on the parametric model of the tissue surface.

In some embodiments, the tool is inserted into a body of a subject via a trocar. In some cases, the method further comprises compensating a location of the tool by identifying an offset caused by an external force applied to the tool via the trocar. In some instances, the offset is determined by comparing a measured 3D coordinates of the tool with a predicted 3D coordinates of the tool.

In some embodiments, the method further comprises determining the optimal path based in part on a cyclic movement of one or more features on the surgical site. In some cases, the cyclic movement is tracked using the image data stream.

Additional aspects and advantages of the present disclosure will become readily apparent to those skilled in the art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 illustrates an autonomous robotic system for performing a surgical procedure, in accordance with some embodiments.

FIG. 2 schematically shows an example of an autonomous robotic system, in accordance with some embodiments.

FIG. 3 shows an example of a camera view, in accordance with some embodiments of the invention.

FIG. 4 shows an example of a plenoptic (i.e., light-field) camera mechanism for capturing images of a surgical scene.

FIG. 5 illustrates an example a free space for a revolute-prismatic joint and a knuckle workspace.

FIG. 6 shows an example of determining knuckle locations.

FIG. 7 shows how focal length of a camera may affect the depth measurement.

FIG. 8 shows an example method for camera calibration, in accordance with some embodiments.

FIG. 9 shows an example of a stitching pattern generated using a stitch prediction algorithm.

FIG. 10 schematically illustrates the alignment of a needle with respect to a tissue surface and a stitching direction.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the embodiments of the present disclosure. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

As used herein, the terms distal and proximal may generally refer to locations referenced from the apparatus, and can be opposite of anatomical references. For example, a distal location of a robotic arm may correspond to a proximal location of an elongate member of the patient, and a proximal location of the robotic arm may correspond to a distal location of the elongate member of the patient.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), a graphic processing unit (GPU), digital signal processors (DSPs), application programming interface (API), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods, algorithms or operations disclosed herein can be implemented in hardware components or combinations of hardware and software such as, for example, ASICs, special purpose computers, or general purpose computers.

### System Overview

As described herein, the present disclosure provides systems and methods for autonomous robotic surgery. In particular, the provided systems and methods may be capable of performing autonomous surgery involving soft tissue. A variety of surgeries or surgical procedures can be performed by the provided system autonomously. The surgeries may include complex in vivo surgical tasks, such as, dissection, suturing, tissues manipulation, and various others. For instance, the provided autonomous robotic system can be controlled through a closed loop architecture using location tracking information (e.g., from visual servoing) as feedback to apply sutures, clips, glue, weld and the like at specified positions.

The surgical tasks can be performed by the autonomous robotic system may be a compound tasks including a plurality of subtasks. For instance, suturing may comprise subtasks such as positioning the needle, biting the tissue, and driving the needle through the tissue. Other surgical tasks such as exposure, dissection, resection and removal of pathology, tumor resection and ablation and the like may also be performed by the autonomous robotic system.

In some embodiments, the provided autonomous robotic system may be utilized in a minimal access surgery (minimally invasive surgery) which minimizes trauma to soft tissue, reduces post-operative pain, promotes earlier mobilization, shortens hospital stays, and speeds rehabilitation. The minimally invasive surgery often requires the use of multiple incisions on a patient's body for insertion of devices therein. Generally, in a minimally invasive surgery, small incisions (usually only millimeters long) are made in the surface of a patient's body, permitting the introduction of probes, scopes and other instruments into the body cavity of the patient. In such kind of surgeries, a number of surgical procedures may be performed autonomously with instruments that are inserted through small incisions in the patient's body (e.g., chest, abdomen, etc.), and supported by robotic arms. The movement of the robotic arms, actuation of end effectors at the end of the robotic arms, and the operations of instruments or tools may be controlled in an autonomous fashion without or with little human intervention.

In some embodiments, the autonomous robotic system may be in the form of a scope such as a laparoscope, an endoscope, a borescope, a videoscope, or a fiberscope. The scope may be optically coupled to an imaging device. When optically coupled with the scope, the imaging device may be configured to obtain one or more images through a hollow inner region of the scope. The imaging device may comprise a camera, a video camera, a three-dimensional (3D) depth camera, a stereo camera, a depth camera, a Red Green Blue Depth (RGB-D) camera, a time-of-flight (TOF) camera, an infrared camera, a charge coupled device (CCD) image sensor, or a complementary metal oxide semiconductor (CMOS) image sensor.

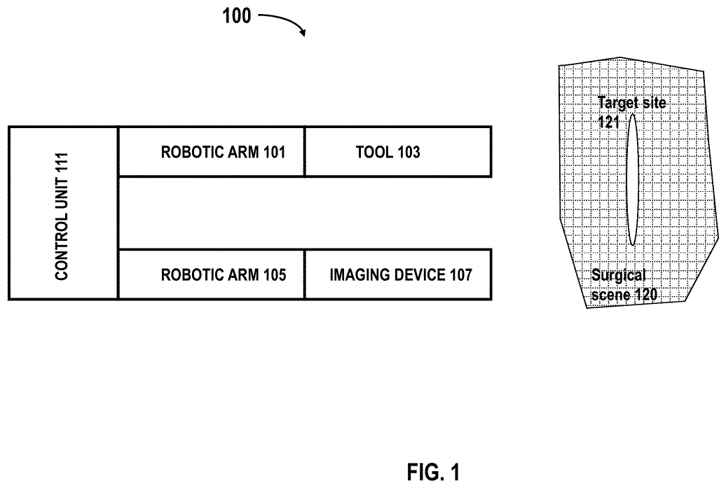

FIG. 1 illustrates an autonomous robotic system 100 for performing a surgical procedure. The surgical procedure may comprise one or more medical operations performed on a surgical site or a surgical scene 120 of a patient. The surgical scene may comprise a target site 121 where a surgical tool 103 may be located to perform the surgical procedure. In some embodiments, the system 100 may comprise a surgical tool 103 and an imaging device 107. The surgical tool 103 and the imaging device 107 may be supported by one or more robotic arms 101, 105. In some cases, the surgical tool 103 and the imaging device 107 may be supported by the same robotic arm. Alternatively, the surgical tool 103 and an imaging device 107 may each be supported by a respective robotic arm (e.g., tool robotic arm 101, camera robotic arm 105).

The imaging device 107 may be configured to obtain one or more images of a surgical scene of a patient. The surgical scene 120 may comprise a portion of an organ of a patient or an anatomical feature or structure within a patient's body. The surgical scene 120 may comprise a surface of a tissue of the patient's body. The surface of the tissue may comprise epithelial tissue, connective tissue, muscle tissue (e.g., skeletal muscle tissue, smooth muscle tissue, and/or cardiac muscle tissue), and/or nerve tissue. The captured images may be processed to obtain location information of the target site 121, the surgical tool or other information (e.g., tissue tension, external force, etc.) for kinematics control and/or dynamics control of the autonomous robotic system.

In some cases, the surgical scene may be a region within a subject (e.g., a human, a child, an adult, a medical patient, a surgical patient, etc.) that may be illuminated by one or more illumination sources. The surgical scene may be a region within the subject's body. In some cases, the surgical scene may correspond to an organ of the subject, a vasculature of the subject, or any anatomical feature or structure of the subject's body. In some cases, the surgical scene may correspond to a portion of an organ, a vasculature, or an anatomical structure of the subject.

In some cases, the surgical scene may be a region on a portion of the subject's body. The region may comprise a portion of an epidermis, a dermis, and/or a hypodermis of the subject. In other cases, the surgical scene may correspond to a wound located on the subject's body. The target site may comprise a wound opening to be sutured close by the autonomous robotic system. Alternatively, the surgical scene may correspond to an amputation site of the subj ect.

In some embodiments, the target site may comprise a target tissue or object to be stitched or connected (e.g., to another target tissue or object) using any of the suturing methods or techniques disclosed herein. The suturing methods and techniques disclosed herein may be used to close a surgical opening (e.g., a slit), attach a first tissue structure to a second tissue structure, stitch a first portion of a tubular structure to a second portion of the tubular structure, stitch a tubular tissue structure to another tissue structure (which may or may not be tubular), stitch a first tissue region to a second tissue region, or stitch one or more tissue flap regions to another tissue structure or tissue region (e.g., a tissue region surrounding the flap region). In some instances, the suturing methods and techniques disclosed herein may be used to perform an arterioarterial anastomosis, a venovenous anastomosis, or an arteriovenous anastomosis.

In some cases, the autonomous robotic system 100 may be used to perform a minimally invasive surgical procedure. At least a portion of the autonomous robotic system (e.g., tool, instrument, imaging device, robotic arm, etc.) may be inserted into the body through an access portal or cannulas. In some cases, access portals are established using trocars in locations to suit the particular surgical procedure. The operations, locations, and movements of the tool may be controlled based at least in part on images captured by the imaging device. In some cases, 2D or 3D images captured by the imaging device, end effector positions and orientations as determined using kinematics of the robotic arms and their sensed joint positions, and tool and camera mechanisms, are calibrated and registered with each other prior to the surgical operation so that the end effector can be controlled autonomously.

The tool 103 may be an instrument selected from a variety of instruments suitable for performing a surgical procedure. For example, the tool can be a stitching or suturing device for performing complex operations such as suturing. Any suitable suturing devices can be utilized for performing autonomous suturing. For example, the suturing device may be a laparoscopic suturing tool. In some cases, the laparoscopic suturing tool may have a mechanism capable of performing soft tissue surgeries such as knot tying, needle insertion, and driving the needle through the tissue or other predefined motions.

The tool 103 may optionally couple to a sensor for sensing stitch tension or tissue tension for the force control. In optional embodiments, a sensor may be operably coupled to the tool for measuring a force or tension applied to the tissue. For instance, a force sensor may be mounted to the tool to measure a force applied to the tissue. In some cases, tension force applied to the tissue may be measured directly using one or more sensors. For instance, sensors such as a magnetic field sensor, a strain gauge, a pressure sensor, a force sensor, an inductive sensor such as, for example, an eddy current sensor, a resistive sensor, a capacitive sensor, an optical sensor, and/or any other suitable sensor, may be configured to measure the suturing force. Alternatively or in addition to, the tension force may be estimated using indirect approach. For instance, an estimation of the length of suturing thread may be calculated. Based on the length of thread and/or angle of the thread, a tension of force in the thread may be calculated which can be used for estimating the force applied to the tissue. In some cases, the measured or estimated force may be used for determining a threshold F. The autonomous robotic system may exit a surgery or procedure if the tension is greater than F for safety.

Similarly, tissue tension may be measured or estimated to determine the threshold force. In some cases, the tissue tension or tissue deformation may be calculated based on the real-time image data. For instance, image data collected by the imaging device may be processed and a geometric surface model of the tissue surface may be obtained. Using the geometric surface model as a smoothness constraint along with the soft tissue modeling (e.g., mass-spring model, motion model, finite element method (FEM), nonlinear FEM, linear or nonlinear elastic 2D/3D simulations, etc.) or other physical constraints (e.g., isometry), the 3D tissue deformation may be estimated and tissue tension may be derived.

The tool 103 may be supported by a robotic arm 101. The robotic arm 101 may be controlled to position and orient the tool with respect to the surgical site 121. In some cases, the tool 103 may be moved, positioned and oriented with respect to the surgical site, by the robotic arm, to perform complex in vivo surgical tasks in an automated fashion. The motion, location, and/or posture of the robotic arm may be tracked using one or more motion sensors or positioning sensors. Examples of the motion sensor or positioning sensor may include an inertial measurement unit (IMU), such as an accelerometer (e.g., a three-axes accelerometer), a gyroscope (e.g., a three-axes gyroscope), or a magnetometer (e.g., a three-axes magnetometer). The IMU may be configured to sense position, orientation, and/or sudden accelerations (lateral, vertical, pitch, roll, and/or yaw, etc.) of (i) at least a portion of the robotic arm or (ii) a tool or instrument that is being manipulated or that is capable of being manipulated using the robotic arm.

Depending on the surgical procedures, the robotic arm and/or the tool may have two, three, four, five, six, seven, eight degree of freedom (DOF) such that the tool is able to be oriented in six degree of freedom (DOF) space. For instance, in the autonomous suturing procedure, the robotic arm 101 may align the tool into an optimal orientation and position the tool at a suturing location (e.g., anchoring point) with respect to a stitching direction and a surface of the tissue thereby minimizing the interaction forces between the tissue and the needle during suturing. In some cases, the robotic arm may be part of a laparoscopic surgical system. Details about the optimal stitching pattern and alignment of the tool are described later herein.

It should be noted that the robotic arm or the tool mechanism can be any mechanism or devices so long as the kinematics are updated according to the robot or tool mechanism. Furthermore, a variety of different surgical tasks or surgical procedures can be performed so long as the path planning and/or trajectory planning of the tool (or end effector) is modified to meet the requirements.

The imaging device 107 may be configured to obtain one or more images of a surgical scene. The imaging device may track the location, position, orientation of the tool and/or one or more features or points of interest on the surgical site in real-time. The captured images may be processed to provide information about a stitch location (e.g., stitch depth) with millimeter or submillimeter accuracy. The depth information and location information may be used for controlling the location, orientation and movement of the tool relative to the target site.

The imaging device 107 can be any suitable device to provide three-dimensional (3D) information about the surgical site. The imaging device may comprise a camera, a video camera, a 3D depth camera, a stereo camera, a depth camera, a Red Green Blue Depth (RGB-D) camera, a time-of-flight (TOF) camera, an infrared camera, a near infrared camera, a charge coupled device (CCD) image sensor, or a complementary metal oxide semiconductor (CMOS) image sensor. The imaging device may be a plenoptic 2D/3D camera, structured light, stereo camera, lidar, or any other camera capable of imaging with depth information. The imaging device may be used in conjunction with passive or active optical approaches (e.g., structured light, computer vision techniques) to extract depth information about the surgical scene. In some cases, the imaging device may be used in conjunction with other types of sensors (e.g., proximity sensor, location sensor, positional sensor, etc.) to provide location information.

The captured image data may be 2D image data, 3D image data, depth map or a combination of any of the above. The captured image data may be processed to obtain location information about at least a portion of the robotic system with respect to the target site and/or depth information of the surgical scene. For instance, 3D coordinates of the tool with respect to the surgical scene may be calculated from the image data. In some instances, plenoptic 3D surface reconstruction of the tissue surface may be calculated, and the location of the tool (e.g., tip location of the instrument) with respect to the 3D surface or 3D coordinates of the tool in the robotic base reference frame may be calculated.

In some cases, the captured image data may be processed to obtain one or more depth maps of the surgical scene. The one or more depth maps may be associated with the one or more images of the surgical scene. The one or more depth maps may comprise an image or an image channel that contains information relating to a distance or a depth of one or more surfaces within the surgical scene from a reference viewpoint. The reference viewpoint may correspond to a location of the imaging device relative to one or more portions of the surgical scene. The one or more depth maps may comprise depth values for a plurality of points or locations within the surgical scene. The one or more depth maps may comprise depth values for a plurality of pixels within the image of the surgical scene. The depth values may correspond to a distance from the imaging device to a plurality of points or locations within the surgical scene. The depth values may correspond to a distance from a virtual viewpoint to a plurality of pixels within an image of the surgical scene. In some cases, the virtual viewpoint may correspond to a position and/or an orientation of the imaging device in real space.

In some embodiments, the imaging device 107 may be supported by a robotic arm 105. The imaging device may provide real-time visual feedback for autonomous control of the tool. In some embodiments, the imaging device 107 and the robotic arm 105 may provide an endoscopic camera to provide a view of the surgical scene. The imaging device may be a 2D articulated camera. In some cases, the camera view may be a 2D view comprising the target site and at least a portion of the tool (e.g., suturing device). Alternatively, the camera view may not comprise an image of the tool while the 3D coordinates of the tool may be calculated based on the kinematic analysis and mechanism of the tool 103, robotic arms 101, 105 and the camera. Details about the 2D camera view and calculation of the 3D coordinates of the tool are described later herein.

The control unit 111 may control the robotic system and surgical operations performed by the tool based at least in part on the real-time visual feedback. For instance, 3D coordinates of the tool and depth information of the surgical scene may be used by the robotic motion control algorithm in open loop or closed-loop architecture. In an autonomous control process, the motion and/or location control feedback loop may be closed in the sensor space. In some cases, the provided control algorithm may be capable of accounting for changes in the dynamic environment such as correcting tool position errors caused by external forces. For instance, errors of tool position may be caused by external forces applied to the robotic arm or the tool through the trocar, and such errors may be calculated and compensated/corrected by updating a kinematic result of the tool. Details about the tool position compensation are described later herein.

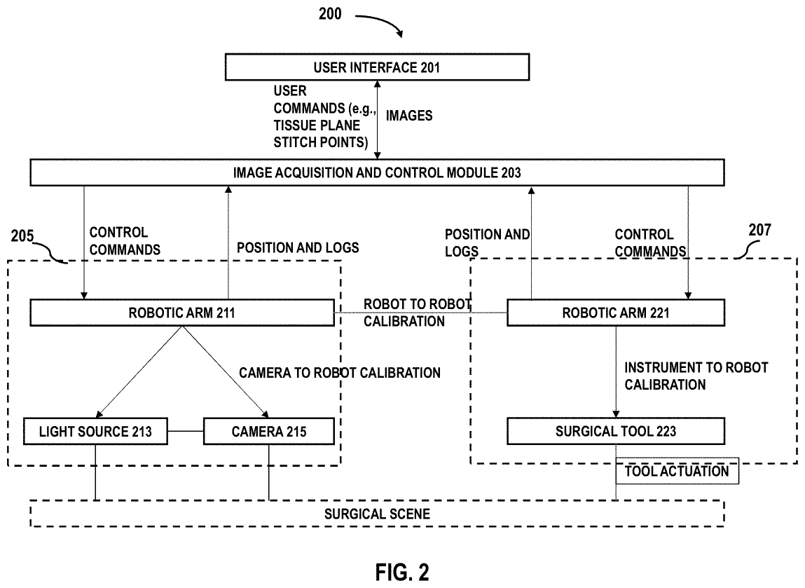

In some embodiments, the autonomous robotic system may perform complex surgical procedures without human intervention. In some embodiments, the autonomous robotic system may provide an autonomous mode and a semi-autonomous mode permitting a user to interact with the robotic system during operation. FIG. 2 schematically shows an example of an autonomous robotic system 200. In some cases, a surgeon may be permitted to interact with the surgical robot as a supervisor, taking over control through a master console whenever required.

In the illustrated example, a surgeon may interact with the autonomous robotic system via a user interface 201. A surgeon may provide commands via the user interface 201 to the image acquisition and control module 203 during the surgical procedures. For instance, the image acquisition and control module 203 may receive user command indicating one or more desired suturing locations on a tissue plane (e.g., a start side of the a wound opening, the end side of the wound opening, and a point to the side of the wound opening, etc.) and the image acquisition and control module 203 may generate a stitching pattern based on the user commands using a stitch prediction algorithm. In another example, a surgeon may be permitted to interrupt and stop a procedure for safety issues. In some cases, real-time images/video and tracking information may be displayed on the user interface.

The user interface 201 may display the acquired visual images overlaid with processed data. For instance, the image acquisition and control module 203 may apply image processing algorithms to detect the tool, and the location of the tool may be tracked and marked in the real-time image data. In another example, the image acquisition and control module 203 may generate an augmented layer comprising augmented information such as the stitching pattern, desired suturing locations with respect to the target site, or other pre-operative information (e.g., a computed tomographic (CT) scan, a magnetic resonance imaging (MRI) scan, or an ultrasonography scan). The augmented layer may be superposed onto the optical view of the optical images or video stream captured by the imaging device, and/or displayed on the display device.

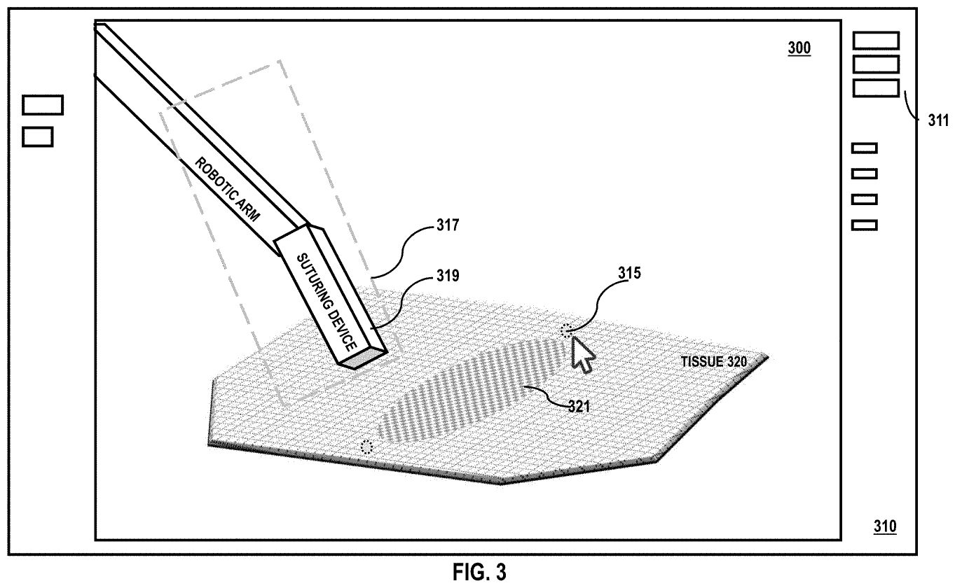

The user interface 201 may include various interactive devices such as touchscreen monitors, joysticks, keyboards and other interactive devices. A user may be able to provide user commands via the user interface using a user input device. The user input device can have any type user interactive component, such as a button, mouse, joystick, trackball, touchpad, pen, image capturing device, motion capture device, microphone, touchscreen, hand-held wrist gimbals, exoskeletal gloves, or other user interaction system such as virtual reality systems, augmented reality systems and the like. Details about the user interface are described with respect to FIG. 3.

In some cases, the image acquisition and control module 203 may receive the location tracking information (e.g., position and logs) from the image-based tracking module 205, combine these with the intraoperative commands from the surgeon, and send appropriate commands to the surgical robot module 207 in real-time in order to control the robotic arm 221 and the surgical tool(s) 223 to obtain a predetermined goal (e.g. autonomous suturing). The depth or location information may be processed by the image-based tracking module 205, the image acquisition and control module 203 or a combination of both.

In some cases, the image acquisition and control module 203 may receive real-time data related to tissue tension, tissue deformation, tension force from the image-based tracking module 205 and/or the surgical robot module 207. The real-time data may be raw sensor data or processed data. In some cases, the image acquisition and control module may be in communication with one or more sensors located at the surgical robot module 207. The one or more sensors may be used for detecting the tension of the suture during the suturing procedure. This can be achieved by monitoring the force required to advance a needle through its firing stroke. Monitoring the force required to pull the suturing material through tissue may indicate stitch tightness and/or suture tension. For example, the one or more sensors may be positioned on the end effector and adapted to operate with the robotic surgical instrument to measure various metrics or derived parameters. The one or more sensors may comprise a magnetic sensor, a magnetic field sensor, a strain gauge, a load cell, a pressure sensor, a force sensor, a torque sensor, an inductive sensor such as an eddy current sensor, a resistive sensor, a capacitive sensor, an optical sensor, and/or any other suitable sensor for measuring one or more parameters of the end effector. Alternatively or in addition to, the tension force may be estimated using indirect approach. For instance, an estimation of the length of suturing thread may be calculated.

Based on the length of thread and/or angle of the thread, a tension of force in the thread may be calculated which can be used for estimating the force applied to the tissue.

In some cases, the measured or estimated force may be used for determining a threshold F for providing safety to the patient or the surgical procedure. For instance, the autonomous robotic system may exit a surgery or procedure if the tension is greater than the threshold F for safety.

In some cases, tissue tension may be measured or estimated to determine the threshold force F. In some cases, the tissue tension or tissue deformation may be calculated based on the real-time image data. For instance, image data collected by the imaging device may be processed and a geometric surface model of the tissue surface may be obtained. Using the geometric surface model as a smoothness constraint along with the soft tissue modeling (e.g., mass-spring model, motion model, finite element method (FEM), nonlinear FEM, linear or nonlinear elastic 2D/3D simulations, etc.) or other physical constraints (e.g., isometry), the 3D tissue deformation may be estimated and tissue tension may be derived. In some cases, tissue tension may be estimated based on the force applied to the tissue. In some cases, tissue tension or deformation may be measured directly using one or more sensors such as a magnetic field sensor, a strain gauge, a pressure sensor, a force sensor, an inductive sensor such as, for example, an eddy current sensor, a resistive sensor, a capacitive sensor, an optical sensor, and/or any other suitable sensor, that are configured to measure tissue compression.

In some cases, the tissue tension or tissue deformation may be calculated and used for controlling the needle motion and/or dynamic control (e.g., force control) of the suturing device. Alternatively or in addition to, the tissue deformation may be minimized by adopting an optimal stitching pattern and tool alignment/trajectory such that the calculation of tissue deformation can be avoided.

The image acquisition and control module 203 may execute one or more algorithms consisted with the methods disclosed herein. For example, the image acquisition and control module 203 may implement a closed loop positioning algorithm, a tool position correction algorithm, for controlling the surgical robot module 207, image processing algorithm and tracking algorithm for tracking the location of the tool or point/feature of interest, surgical operation algorithm (e.g., stitch prediction algorithm) to generate a stitching path for path planning for the tool, and various other algorithms. One or more of the algorithms may be applied to the real-time image data to generate the desired information. For example, the image acquisition and control module 203 may execute the tool position correction algorithm to correct an error in tool position caused by an external force based at least in part on the image data.

In some embodiments, one or more of the aforementioned algorithms may require kinematic analysis of the robotic system. For instance, the forward and/or inverse kinematics of the robotic system may be solved and tested by the robot to robot calibration between the two robotic arms 211, 221, camera to robot calibration between the camera 215 and the robotic arm 211, the instrument and robot calibration between the surgical tool 223 and the robotic arm 221, and the mechanism of the surgical tool 223. For instance, the location tracking algorithm may process the image data to generate the location of the surgical tool without using image segmentation. The location of the surgical tool with respect to a surgical site may be calculated by projecting the tool into the camera's coordinate space based on the kinematic analysis between the tool and the camera (e.g., transformations from the surgical tool to the surgical tool flange to the surgical tool base to the camera base to the camera flange to the camera). In another example, the tool position correction algorithm may be applied to the image data to output a correction of the position error due to an external force exerted onto the robotic system such as the surgical tool module. The correction may be obtained by measuring an offset between the expected point location of an instrument tip (or other feature of the instrument) and the actual location of the instrument tip, and calculating an affine transformation based on the kinematic analysis/transformation matrix between the instrument and the camera frames. Details about the location tracking algorithm and the tool position correction algorithm are described later herein.

The image acquisition and control module 203 may be implemented as a controller or one or more processors. The image acquisition and control module may be implemented in software, hardware or a combination of both. The image acquisition and control module 203 may be in communication with one or more sensors (e.g., imaging sensor, force sensor, positional/location sensors disposed at the robotic arms, imaging device or surgical tool) of the autonomous robotic system 200, a user console (e.g., display device providing the UI) or in communication with other external devices. The communication may be wired communication, wireless communication or a combination of both. In some cases, the communication may be wireless communication. For example, the wireless communications may include Wi-Fi, radio communications, Bluetooth, IR communications, or other types of direct communications.

In some embodiments, the image-based tracking module 205 may comprise an imaging device 215 supported by a robotic arm 211. The imaging device and the robotic arm can be the same as those described in FIG. 1.

In some embodiments, the image-based tracking module 205 may comprise a light source 213 to provided illumination light. The wavelength of the illumination light can be in any suitable range and the light source can be any suitable type (e.g., laser, LED, fluorescent, etc.) depending on the detection mechanism of the camera 215.

The light source and the camera may be selected based on the optical approach or optical techniques used for obtaining the depth information of the surgical scene. The provided robotic system may adopt any suitable optical techniques to obtain the 3D or depth information of the tool and the surgical scene. For example, the depth information or 3D surface reconstruction may be achieved using passive methods that only require images, or active methods that require controlled light to be projected into the surgical site. Passive methods may include, for example, stereoscopy, monocular shape-from-motion, shape-from-shading, and Simultaneous Localization and Mapping (SLAM) and active methods may include, for example structured light and Time-of-Flight (ToF). In some cases, computer vision techniques such as optical flow, computational stereo approaches, iterative method combined with predictive models, machine learning approaches, predictive filtering or any non-rigid registration methods may be used to continuously track soft tissue location and deformation or to account for changing morphology of the organs.

The light source 213 may be located at the distal end of the robotic arm 211. Alternatively or in addition to, illumination light may be provided by fiber cables that transfer the light of the light source 213 located at the proximal end the robotic arm2 11, to the distal end of the robotic arm (endoscope).

In some cases, the camera 215 may be a video camera. The camera can be the same as the imaging device as described in FIG. 1. The camera may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). In some cases, the image sensor may be provided on a circuit board. The circuit board may be an imaging printed circuit board (PCB). The PCB may comprise a plurality of electronic elements for processing the image signal. For instance, the circuit for a CCD sensor may comprise AID converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) that can be further processed by a camera circuit or processors of the camera. In some cases, the image sensor may comprise an array of optical sensors.

In some cases, the camera 215 may be a plenoptic camera having a main lens and additional micro lens array (MLA). The plenoptic camera model may be used to calculate a depth map of the captured image data. In some cases, the image data captured by the camera may be grayscale image with depth information at each pixel coordinate (i.e., depth map). The camera may be calibrated such that intrinsic camera parameters such as focal length, focus distance, distance between the MLA and image sensor, pixel size and the like are obtained for improving the depth measurement accuracy. Other parameters such as distortion coefficients may also be calibrated to rectify the image for metric depth measurement. The depth measurement may then be used for controlling the robotic arm and/or the surgical robotic module.

As described above, the camera 215 may perform pre-processing of the capture image data. In an embodiment, the pre-processing algorithm can include image processing algorithms, such as image smoothing, to mitigate the effect of sensor noise, or image histogram equalization to enhance the pixel intensity values. In some cases, one or more processors of the image-based tracking module 205 may use optical approaches as described elsewhere herein to reconstruct a 3D surface of the tissue or a feature of the tissue (e.g., wound opening, open slit to be sutured), and/or generate a depth map of the surgical scene. For instance, an application programming interface (API) of the image-based tracking module 205 may output a focused image with depth map. Alternatively, the depth map may be generated by one or more processors of the image acquisition and control module 203.

In some cases, the power to the camera 215 or the light source 213 may be provided by a wired cable. In some cases, real-time images or video of the tissue or organ may be transmitted to external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the laparoscope to the processors situated at the base of the robotic system via wires, copper wires, or via any other suitable means.

Tool projection and location tracking algorithm

In some embodiments, passive optical techniques may be used for generating the depth map, tracking tissue location and/or tool location. As described above, the depth information or 3D coordinates of the tool with respect to a tissue surface may be obtained from the captured real-time image data. In some embodiments, the provided location tracking algorithm may be used to process the image data to obtain the 3D coordinates of the surgical tool using a model-based approach without image segmentation. For example, the location of the surgical tool with respect to a tissue surface or the 3D coordinates of the surgical tool may be calculated by projecting the surgical tool into the camera's coordinate space based on the kinematic analysis between the tool and the camera reference frames. The provided location tracking algorithm may be robust to outliers, partial occlusions, changes in illumination, scale and rotation thereby providing additional safety and reliability to the system. This may be beneficial to cope with a dynamic and deformable environment (e.g., in a laparoscopic surgery). For instance, when the illumination is not available or when the surgical tool is not recognizable in the image data (e.g., presence of specular highlights, smoke, and blood in laparoscopic intervention, occlusion of the camera, obstructions come into view, etc.), 3D location of the tool can still be tracked to ensure patient safety without relying on image segmentation of the tool in the camera view. For instance, a user may be permitted to view a marker indicating the location of the surgical tool in the camera view (e.g., 2D laparoscopic image) without the presence of the surgical tool in the image.

The location tracking algorithm may comprise projecting the surgical tool into the camera's coordinate space. The locating tracking may be achieved based on the kinematic analysis between the tool and the camera so that the tool coordinates can be projected to the camera reference frame. For instance, a tool may be coupled to a tool flange which is linked to a tool robot base, the tool robot base is linked to the camera robot base which is linked to the camera through the camera flange. Based on the predefined dimensions, models and mechanism of the tool, the tool flange, the tool robotic arm, the camera robotic arm, the camera and the robotic system (robot to robot relationship), transformations from the tool to the tool flange to the tool robotic base to the camera robotic base to the camera flange to the camera can be calculated. The coordinates of the tool in the camera view can be determined based on the transformation. In some cases, calibration and registration one or more components of the system such as the camera, tool, robotic arms may be performed at an initial stage prior to the surgical procedure. The locating tracking algorithm may also be used for other purposes such as for determining if a tracked piece of tissue is being occluded by the tool.

FIG. 3 shows an example of a camera view 300, in accordance with some embodiments of the invention. In the illustrated example, the camera view 300 may be a 2D laparoscopic image of a surgical scene. A location 317 of a surgical tool in the camera view may be displayed without requiring the presence of the surgical tool in the optical view of the optical images. The surgical scene may comprise a target site 320 such as portion of an organ of a patient or an anatomical feature or structure within a patient's body. As described above, the surgical scene may comprise a surface of a tissue of the patient's body. The surface of the tissue may comprise epithelial tissue, connective tissue, muscle tissue (e.g., skeletal muscle tissue, smooth muscle tissue, and/or cardiac muscle tissue), and/or nerve tissue. In some cases, a reconstructed three-dimensional (3D) tissue surface or a depth map of the surgical scene may be obtained from the images of the surgical scene.

In some cases, the surgical scene may be a region on a portion of the subject's body. The region may comprise a portion of an epidermis, a dermis, and/or a hypodermis of the subject. In the illustrated example, the surgical scene may comprise a feature such as a wound opening 321 or other locations where the surgical tasks to be performed.

In some cases, the camera view or the surgical scene may comprise the target site 320 and at least a portion of the surgical tool (e.g., suturing device) 319. Alternatively, the surgical tool may not be visible in the optical view of the optical images. The location of the surgical tool may be calculated using the location tracking algorithm as described above. In some cases, the location of surgical tool may be marked in the image to augment the image data.

In some cases, the one or more images of the surgical scene may comprise a superimposed image. The superimposed image may comprise an augmented layer including augmented information such as the graphical element 317 indicating the location of the surgical tool. In some cases, the augmented layer may comprise one or more graphical elements representing a stitching pattern, one or more desired suturing locations 315 with respect to the target site. The augmented layer may be superposed onto the optical view of the optical images or video stream captured by the imaging device, and/or displayed on a display device. The augmented layer may be a substantially transparent image layer comprising one or more graphical elements (e.g., box, arrow, etc.). The transparency of the augmented layer allows the optical image to be viewed by a user with graphical elements overlay on top of the optical image.

The one or more elements in the augmented layer may be automatically generated by the autonomous robotic system or based on a user input. For instance, a wound opening 321 may be segmented from the image data and graphical markers indicating the location of the wound opening may be generated in the augmented layer. As described above, the image acquisition and control module may employ various optical techniques (e.g., images and edge detection techniques) to track a surgical site where a surgical instrument is used to complete a surgical task. In some cases, the location (e.g., wound opening 321, wound slit, etc.) where the surgical instrument is to perform a surgical task may be identified automatically by the image acquisition and control module. For instance, the wound opening 321 may be segmented and one or more desired/user-selected suturing locations 315 may be overlaid on the real time images with respect to the wound opening. Alternatively or in addition to, the location where the surgical instrument is to perform a surgical task may be determined based at least in part on user provided command such as the one or more user-selected suturing locations 315. In some cases, graphical markers 315 representing a user selected suturing location/point may be overlaid onto the real time images. The coordinate of the graphical markers in the camera reference frame may be calculated and updated in real-time which may allow operators or users to visualize the accurate location of the tool moving with respect to the user selected suturing locations.

The superimposed image may be real-time images rendered on a graphical user interface (GUI) 310. The GUI may be provided on a display. The display may or may not be a touchscreen. The display may be a light-emitting diode (LED) screen, organic light-emitting diode (OLED) screen, liquid crystal display (LCD) screen, plasma screen, or any other type of screen. The display may be configured to provide a graphical user interface (GUI) rendered through a software application (e.g., via an application programming interface (API) executed on the system). This may include various devices such as touchscreen monitors, joysticks, keyboards and other interactive devices. In some embodiments, a user may be able to provide user commands using a user input device. The user input device can have any type of user interactive component, such as a button, mouse, joystick, trackball, touchpad, pen, image capturing device, motion capture device, microphone, touchscreen, hand-held wrist gimbals, exoskeletal gloves, or other user interaction system such as virtual reality systems, augmented reality systems and the like.

As illustrated in the example, a user may input a desired suturing location 315 by clicking on the image. The coordinates of the suturing location may be expressed in the camera frame. The coordinates of the suturing location may then be transformed into the tool robot base frame to generate the corresponding (Cartesian) robotic/tool motions. This transformation may be achieved using camera registration and calibration as described later herein. In some cases, the 3D coordinates of the suturing location may also be used to generate a stitching pattern. Details about the stitching pattern generation and stitch prediction algorithm are described later herein. A graphical marker representing the suturing location on a tissue surface plane may be generated and the graphical marker may be overlaid onto the real-time image or video such that the location of the graphical marker may be updated on the display.

In some embodiments, the GUI may also provide a master console allowing a user to take over control of the autonomous robotic system. For example, a user may be permitted to select a surgical procedure to be performed, select a surgical tool, initiate/stop a surgical procedure, or modify other parameters by interacting with one or more graphical elements 311 provided within the GUI.

### Camera Calibration and Registration

In some embodiments, the imaging device may be a 3D imaging device of a standard laparoscope system configured to capture image data of a surgical scene. In some cases, one or more depth maps of the surgical scene may be generated.

The one or more depth maps may be associated with the one or more images of the surgical scene. The one or more depth maps may comprise an image or an image channel that contains information relating to a distance or a depth of one or more surfaces within the surgical scene from a reference viewpoint. The reference viewpoint may correspond to a location of the imaging device relative to one or more portions of the surgical scene. The one or more depth maps may comprise depth values for a plurality of points or locations within the surgical scene. The one or more depth maps may comprise depth values for a plurality of pixels within the image of the surgical scene. The depth values may correspond to a distance from the imaging device to a plurality of points or locations within the surgical scene. The depth values may correspond to a distance from a virtual viewpoint to a plurality of pixels within an image of the surgical scene. In some cases, the virtual viewpoint may correspond to a position and/or an orientation of the imaging device in real 3D space.

The provided autonomous robotic system and location tracking algorithm may achieve real-time location tracking with sub-millimeter accuracy. For example, the image data may be processed and depth map may be generated in real-time at a speed greater than or equal to 1 frame per second (fps), 2 fps, 5 fps, 10 fps, 20 fps, 30 fps, 40 fps, 50 fps at resolution greater than or equal to about 352×420 pixels, 480×320 pixels, 720×480 pixels, 1280×720 pixels, 1440×1080 pixels, 1920×1080 pixels, 2008×1508 pixels 2048×1080 pixels, 3840×2160 pixels, 4096×2160 pixels, 7680×4320 pixels, or 15360×8640 pixels.

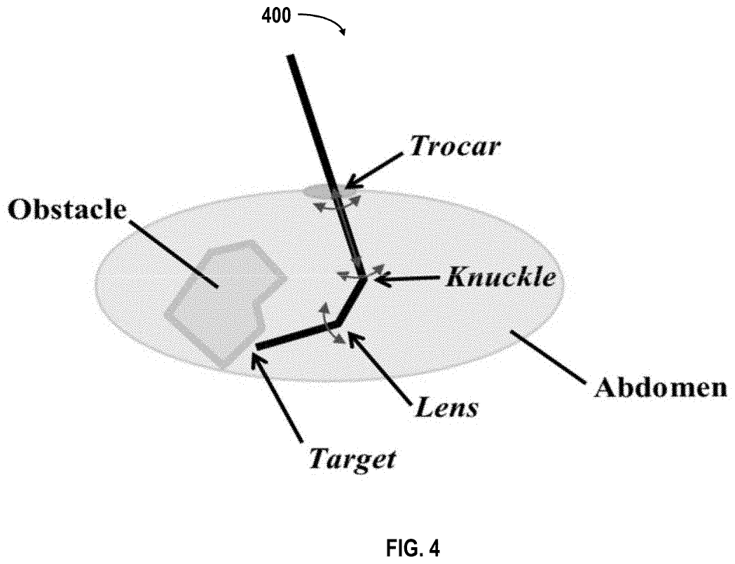

The term camera registration may generally refer to the alignment of the camera frame to the robotic system (e.g., real 3D space). For example, camera registration may comprise determining the relationship between camera's 3D coordinates and camera robot base (e.g., flange of the camera robotic arm). This is needed for determining the relationship between the coordinates of a location in a camera reference frame and the coordinates of the location in the robot reference frame. FIG. 4 shows an example of a plenoptic camera (i.e., light-field camera) mechanism 400 for capturing images of a surgical scene. As shown in the example, the camera is supported by a camera robotic arm and is inserted towards the surgical scene through a trocar. The camera mechanism may be 3-DOF including trocar (revolute), insertion (prismatic) and knuckle (revolute). The camera model or camera work space may then be obtained by establishing a free-object space (Sₖ) for the knuckle including computing a proximal free-object space (Sₚᵣₒₓ) around the trocar and a distal free-object space (S_{dist}) about the target.

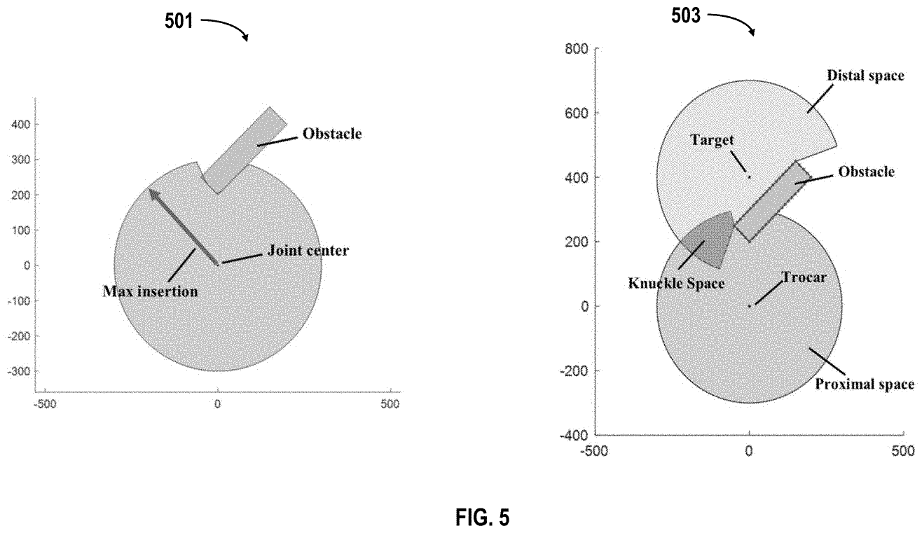

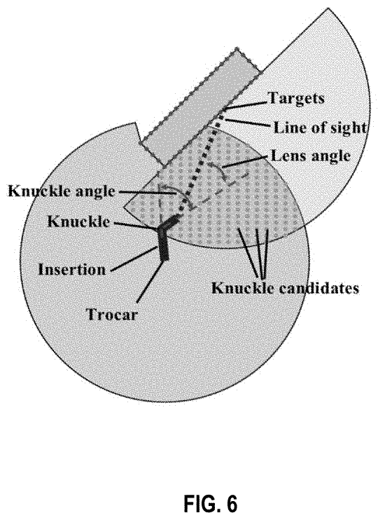

FIG. 5 illustrates an example a free space 501 for the revolute-prismatic joint and a knuckle workspace 503. For example, once the proximal and distal intersection for a given target are established, the intersection may define possible knuckle workspace comprising a set of candidate points. The set of candidate points for the knuckle location can guarantee collision avoidance. As shown in FIG. 6, a knuckle location may be determined from the set of candidate points while satisfying the knuckle joint range. For example, inverse kinematics are solved for the set of candidate points until the first valid solution (i.e., satisfying the knuckle joint range) is found, then the valid solution is determined to be the knuckle angle.

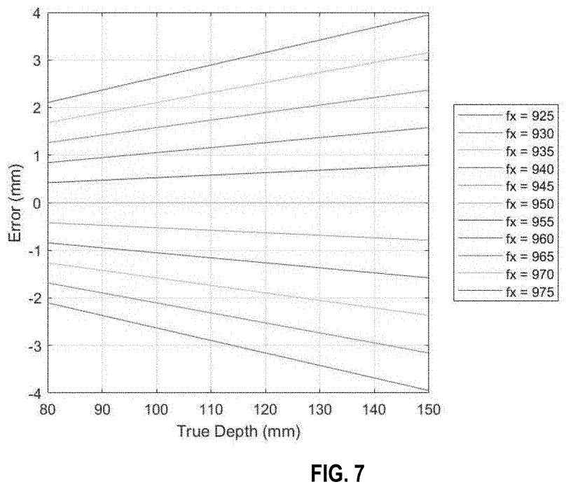

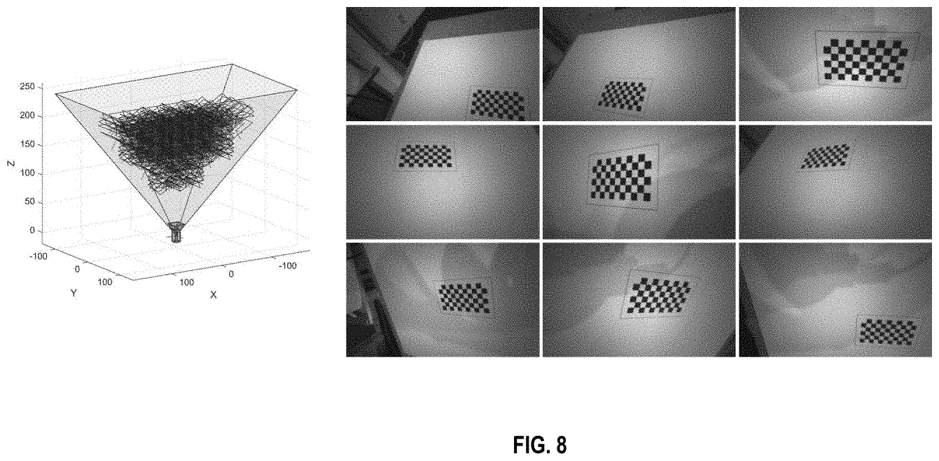

Camera calibration may be performed to improve the camera registration accuracy. The provided camera calibration method may provide the intrinsic parameters of the camera (e.g., focal length, principal point, lens distortion, etc.) with improved measurement accuracy. FIG. 7 shows how focal length may affect the depth measurement. The camera calibration process can use any suitable method. FIG. 8 shows an example method for camera calibration. In the illustrated example, recognizable patterns (e.g., checkerboards) with known or unknown locations relative to the camera robot's base may be used. The camera is positioned into a variety of different point of views with respect to the patterns and/or the pattern may be positioned into different positions/orientations with respect to the camera. Images of the pattern are captured along with the corresponding camera robot's configuration. 3D coordinates of multiple points on the pattern are solved from the image. The process may be repeated multiple times on the same pattern. In some cases, the process may be repeated using different patterns. The camera view is then calibrated which translates the depth and XY locations of the points on the pattern to metric 3D measurements. Using the data of the robot's configuration and the camera 3D points, a transformation between the camera and the flange of the camera robotic arm is obtained. Any suitable mathematic techniques (e.g., least squares matrix solution approach) can be adopted to determine the transformation.

### Stitch Prediction Algorithm

In some embodiments, the tool may move along a surgical operation path to perform surgical operations. In some cases, tool trajectories during the surgical operation may be generated based on the surgical operation path.

In the case of suturing, the surgical operation path may comprise a stitching pattern. The stitching pattern may be generated using a stich prediction algorithm. In some cases, the stitching pattern may be updated dynamically according to the complex environment such as the dynamic deformation of the tissue, changes in the location and/or shape of the tracked target site (e.g., wound opening) and the like. In some cases, the stitching pattern may comprise a series of anchoring points and the coordinates of the series of anchoring points in the 3D space may be used to generate control commands to effectuate the movement and operation of the surgical tool. In some cases, the location of the anchoring points may be updated automatically to adapt to unpredictable changes such as non-rigid deformation of the tissue as a result of suturing.

In some cases, the stitching pattern may comprise a pattern with one or more segments. The one or more segments may comprise one or more linear or substantially linear segments. In some cases, the one or more segments may not or need not be linear or substantially linear. The one or more segments may be used to secure two or more tissue structures or tissue regions together via one or more anchoring points located on or near the two or more tissue structures or tissue regions. The stitching pattern may be any suitable pattern for closing a surgical opening (e.g., a slit), attaching a first tissue structure to a second tissue structure, stitching a first portion of a tubular structure to a second portion of the tubular structure, stitching a tubular tissue structure to another tissue structure (which may or may not be tubular), stitching a first tissue region to a second tissue region, or stitching one or more tissue flap regions to another tissue structure or tissue region (e.g., a tissue region surrounding the flap region).

The stitching pattern may be generated autonomously or semi-autonomously. In some cases, the stitching pattern may be generated autonomously without user intervention. For instance, a wound opening may be identified in the captured image data and the stitching pattern may be generated using a predefined algorithm. Alternatively or in addition to, the stitching pattern may be generated based at least in part on user input data (e.g., user selected/desired suturing location).

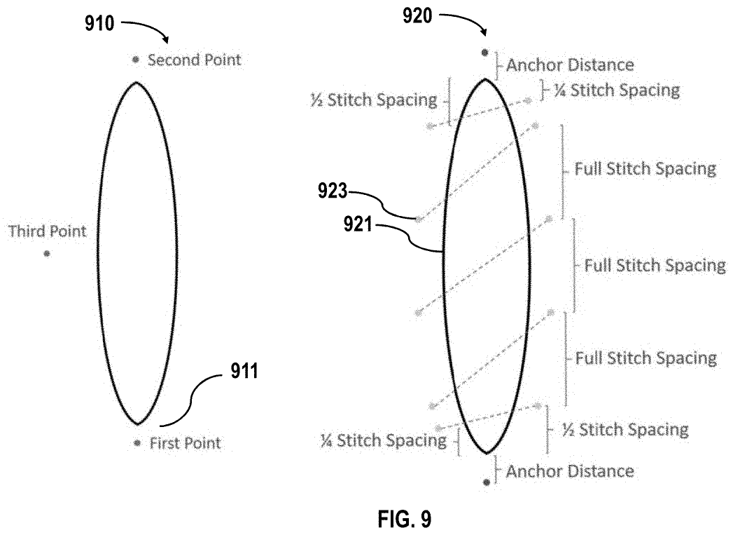

FIG. 9 shows an example of a stitching pattern generated using the provided stitch prediction algorithm. As described above, in some cases, a user may be permitted to provide user command indicating one or more desired suturing locations. Using 3D data obtained from the image-based tracking module and one or more user-inputted suturing locations, the stitch prediction algorithm may automatically determine where to place stitches in order to suture an open slit or wound opening.

In the illustrated example 910, a user may select one or more desired locations 911 for performing suturing. In some cases, the one or more locations may be selected in an order corresponding to the start location and end location of the closure. For example, the first point, second point and third point shown in the example 910 may be located at the start side of the slit, the end side of the slit, and to the side of the slit corresponding to the start location, end location and auxiliary location of the stitching pattern. Any number of locations can be provided. In some cases, the one or more locations may generally indicate a rough dimension (e.g., width, length, etc.) of the stitching pattern.

The one or more suturing locations may be received via a GUI (e.g., the GUI described in FIG. 3). The coordinates of the suturing location may be expressed in the camera frame. Next, the coordinates of the suturing location may be transformed into the tool robot base frame to generate the corresponding (Cartesian) robotic/tool motions. This transformation may be achieved using camera registration and calibration as described elsewhere herein. In some cases, upon receiving the one or more desired suturing locations, the stitch prediction algorithm may automatically generate a stitching pattern comprising a series of metric positions with respect to the 3D metric tissue surface. The series of metric positions may be used to perform path planning for the end effector of the surgical tool module, trajectory planning for the needle/tool, or generate control commands to effect the position, orientation and movement of the needle/tootle.

In an example stitch prediction process 920, the stitch prediction algorithm comprises the following steps:

1. Fit a quadratic equation to the metric 3D surface of the tissue outside of the wound opening or open slit 921. Use the quadratic surface equation to recalculate the metric 3D surface of the tissue in the image to smooth out missing data and extrapolate the surface over the open slit approximating a closure state of the wound opening.

2. Calculate the location of the anchoring points 923 of the running stitch.

2.1 Rotate the image so the contour axis specified by the first two user-specified location points is aligned into a horizontal line centered in the image.

2.2 Extract the centerline of the contour and the corresponding metric surface depth information.

2.3 Use the grayscale intensity value of the centerline to identify the edge points of the open slit. From the edge points, the extrapolated metric data is used to offset the edge points along the centerline according to a specified anchor distance.

2.4 Search the pixels in a 2D area around the edge points of the slit to find the actual contour axis. Repeat 2.1-2.4 until the contour axis converges.

2.5 Use a specified stitch spacing to determine the number of evenly spaced stitches needed to close the open slit.

3. Each stitch consists of a point on each side of the open slit. In the direction of stitching, the first point is on the left side of the slit and offset from the second point on the right. This is designed to prevent previous stitches from interfering with the following stitches.

3.1 Calculate the locations of the stitches along an axis of the open slit.

3.2 Using the same process 2.1-2.3 to find the locations of the left and right sides of the stitch.

It should be noted that depending on the wound type and/or specific surgical procedures, various suturing techniques can be adopted. For example, the suturing techniques may be running stitches or interrupted sutures. The provided stitch prediction algorithm may account for the closure state of the wound and a stitch between a pair of stitch points may be independent of the previous stitches.

### Tool Alignment

In some embodiments, the tool such as a suturing needle may be aligned to an optimal orientation and is positioned to a location relative to the tissue surface to minimize the stress on the tissue. For instance, the suturing needle may be positioned at an anchoring point, a needle plane may be rotated to be aligned with a stitching direction, and the suturing needle may be inserted into the tissue surface orthogonally thereby minimizing the interaction forces between the tissue and the suturing needle during suturing.

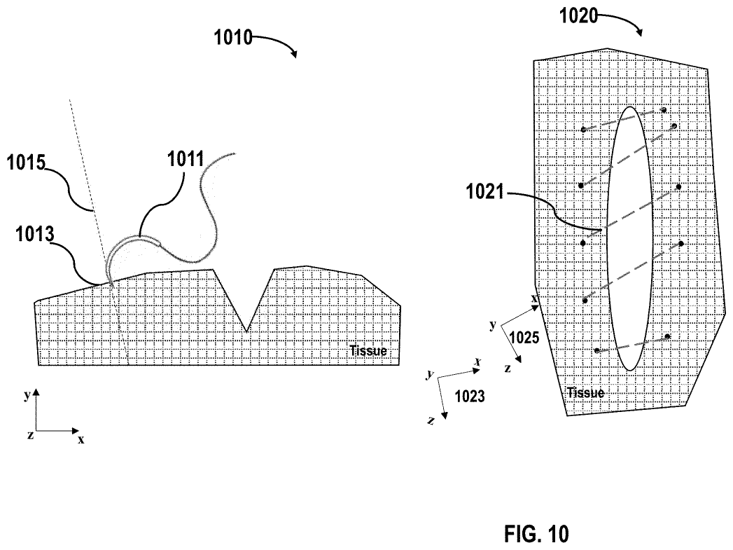

FIG. 10 schematically illustrates alignment of a needle with respect to a tissue surface 1013 and a stitching direction 1021. In some cases, the needle 1011 may be inserted into the tissue orthogonally and the tool plane may be aligned with the stitching direction to minimize the stress on the tissue.

In some embodiments, the suturing device may have predefined motion for moving the needle. For example, a suture head assembly may house a mechanism for driving a curved needle in a complete 360-degree circular arc. The orientation of the suture head assembly is designed such that when the needle 1011 is attached to the suture head assembly the needle 1011 is driven in a curved path about an axis approximately perpendicular to the longitudinal axis of the suturing device. The needle 1011 is in a needle plane (e.g., XY plane) parallel to the drive mechanism and fits into the same space in the suture head assembly. The tool model may be predefined such that the alignment of the needle can be controlled by aligning the suturing device/tool.

As shown in a cross-sectional view 1010 (parallel to the needle plane), the optimal approach angle 1015 may be perpendicular to the tissue surface 1013 and as shown in the top view 1020 (perpendicular to the needle plane), the needle plane may be rotated/oriented (e.g., rotated from a first stitching direction orientation 1023 to a second stitching direction orientation 1025) to be aligned to the stitching direction.

In some cases, the optimal insertion angle 1015 may be obtained by first determining an anchoring point using the stitch prediction algorithm, fitting a quadratic equation to the local tissue surface data surrounding the anchoring point, and using the quadratic surface equation to recalculate the metric 3D surface of the tissue in the local tissue surface area to smooth out missing data and extrapolate the surface over any irregularities. Next, a plane can then be fit to the local tissue surface area, yielding a normal vector to the plane/local surface area. The stitching direction 1021 is determined by the stitch prediction algorithm as described above, and the tool plane defined in the tool model may then be aligned to be parallel to the stitching direction. For instance, the stitching direction may be transformed from the camera space to the tool robot base coordinates and is used to generate control commands to orient the tool.

### Tool Position Correction

In some embodiments, at least a portion of the autonomous robotic system (e.g., the surgical tool module, camera, robotic arm, etc.) may be inserted into a patient body through an access portal or cannulas. In some cases, access portals are established using trocars in locations to suit the particular surgical procedure. In some situations, external forces may be exerted onto the surgical tool by the trocar due to the relative motion between the tool and the patient's body. Such external force may cause errors in tool position. Such errors may be calculated and compensated/corrected using a tool position correction algorithm.

In some embodiments, the effect of the external force may be modeled as an additional affine transform applied to the transformation between the tool model and the flange of the tool robotic arm. The affine transform representing the external trocar forces may be obtained by measuring an offset between the expected point location of an instrument tip and the actual location of the instrument tip. The affine transformation can be calculated based on the kinematic analysis between the tool and the camera frame. For instance, from the 2D camera view, the location of features on the distal end of the tool can be identified. With the associated depth information, the feature locations in the metric 3D space can be determined. Predicted 3D locations of the features are also calculated using the model-based approach (e.g., based on the base to flange transform of the robotic arm and the static tool model). By comparing the locations in the metric 3D space with the predicted 3D locations of the features, the affine transform representing the external trocar forces can be calculated. The same algorithm can be used to correct any external forces exerted onto the robotic system.

The affine transform representing the external forces may be calculated and updated in real-time. To correct the tool position, the affine transform may be applied to the kinematics model and update the kinematics analysis result during the surgical procedure.

Below is an example of an affine transform that is applied to the kinematic model:

H_{trocar correction}*H_{flange-to-instrument}*H_{base-to-flange}*H_{base-to-base}*H_{flange-to-robot-base}*H_{camera-to-flange}* (DesiredPointInCameraSpace)

It should be noted that the transform for correcting the tool position can be applied to any suitable location of the kinematics model. For example, depending on where the external force is exerted onto, the correction transform matrix can be applied to correct errors in the base-to-base calculation or the camera-to-flange calculation.

### Tracking POI During Procedure

The autonomous robotic system may be capable of tracking a user specified point of interest or feature of interest during a surgery. For instance, the provided tracking algorithm may be used to track a respiratory motion of the patient which can be used for planning the surgical tasks. For instance, the respiratory motion of the patient may be regulated during surgeries. The cyclic motion may be tracked and a respiratory motion model may be built. The respiratory motion model may be used for planning tool trajectories and planning the surgical tasks (e.g., suturing). For example, it is beneficial to time surgical tasks (e.g., suturing) or subtasks (e.g., inserting needle) for the pause between exhaling and inhaling.

The oscillation motion of the POI can be used to characterize and build the respiratory motion model by tracking the tissue surface, internal anatomical landmarks or other user specified points of interest (POI) in the 3D metric space. For instance, parameters such as the length of a breath, the amplitude of motion, and the placement and length of the pause within the breathing motion can be calculated. The respiratory motion or other regulated motion of the surgical site can be characterized by tracking the location of the POI which may be performed autonomously without user intervention. For example, the respiratory motion model may be calculated and updated as new image data processed and the updated respiratory motion model may be used for tool trajectory planning or other purposes as described above.

### EXAMPLE

In some cases, the systems and methods disclosed herein may be used for fully autonomous, endoscopic robot-assisted closure of a ventral hernia. The provided autonomous or automated functions may enhance a surgeon's technical and cognitive capabilities in surgery to improve clinical outcomes and safety. For example, complex surgical tasks such as intestinal anastomosis may be performed autonomously in open surgery using the systems and methods provided herein. In an experiment, the systems disclosed herein were used to perform in vivo and ex vivo robot-assisted laparoscopic, fully autonomous ventral hernia repairs in various models, including a phantom model and a preclinical porcine model.

The system utilized in the experiment comprises two portable robotic arm subsystems comprising an off-the-shelf seven-DOF arm on a mobile cart with a one-DOF suturing tool end effector on the first arm and a proprietary 3-D camera on the second arm. A simple, user-friendly registration workflow supports a quick setup of portable, bed-side robotic systems. Improved proprietary tracking algorithms for motion and deformable soft tissue models based on the OpenCV CUDA implementation of Oriented FAST and Rotated BRIEF (ORB) track at least four arbitrary points on a deformable tissue in real-time without using fiducials or biomarkers, and provides real-time adjustments to the suture plan in ex vivo and in vivo procedures. For the in vivo feasibility study, a 10-cm length full thickness incision on the inner left lateral abdominal wall of a pig was used to mimic a clinical ventral hernia model.

### Result

The 3-D laparoscope used for the procedure comprises a chip-on-tip stereo camera with a camera housing. The camera housing may have a dimension of at most about 22.7 mm×23.2 mm×111.8 mm. The 3-D laparoscope provides depth images at 30 fps with a 65% fill ratio (which fill ratio corresponds to the percentage of pixels with valid depth), and a temporal noise of about 2.61 mm when looking at a target about 80 mm (working distance) away from the camera sensors. The 3-D camera computes the depth of a tracked point after time-averaging a plurality of frames (e.g., 5 frames), which can result in a decrease in temporal noise. The modified suture algorithms included such variables as preset inter-suture distance and width from tissue edge for a given tissue thickness, and resulted in reducing inter-suture variances. The suturing methods used also reduced mean completion time per suture. The systems of the present disclosure were successfully used to generate a suture plan, detect deformations during the procedure, automatically adjust the suture plan to correct for the unstructured motions, and execute the updated suture plan to permit clinically acceptable closure of the ventral hernia.

The experiment successfully demonstrates an in vivo and ex vivo laparoscopic, robot-assisted, fully autonomous ventral hernia repair in various models, including a phantom model and a preclinical porcine model. In addition, the experiment shows the ability to generate one or more 3-D point clouds without cumbersome fluorophore markers and with additional improvements on the form factor, computer vision algorithms, real time 3-D tracking capabilities, and suturing algorithms.

### Computer Systems

Another aspect of the present disclosure provides computer systems that are programmed or otherwise configured to implement methods of the disclosure. One or more processors may be used to implement the various algorithms and the image-based robotic control systems of the present disclosure. The processor may be a hardware processor such as a central processing unit (CPU), a graphic processing unit (GPU), a general-purpose processing unit, or computing platform. The processor may be comprised of any of a variety of suitable integrated circuits, microprocessors, logic devices and the like. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, 16 bit, or 8 bit data operations.

The processor may be a processing unit of a computer system. The processors or the computer system used for camera registration and calibration and other pre-operative algorithms may or may not be the same processors or system used for implementing the control system. The computer system can be an electronic device of a user or a computer system that is remotely located with respect to the electronic device. The electronic device can be a mobile electronic device.

The computer system can be operatively coupled to a computer network (“network”) with the aid of a communication interface. The network can be the Internet, an intranet and/or extranet, an intranet and/or extranet that is in communication with the Internet, or a local area network. The network in some cases is a telecommunication and/or data network. The network can include one or more computer servers, which can enable distributed computing, such as cloud computing. In some instances, the machine learning architecture is linked to, and makes use of, data and stored parameters that are stored in cloud-based database. The network, in some cases with the aid of the computer system, can implement a peer-to-peer network, which may enable devices coupled to the computer system to behave as a client or a server.

The computer system can comprise a mobile phone, a tablet, a wearable device, a laptop computer, a desktop computer, a central server, etc. The computer system includes a central processing unit (CPU, also “processor” and “computer processor” herein), which can be a single core or multi core processor, or a plurality of processors for parallel processing. The CPU can be the processor as described above.

The computer system also includes memory or memory locations (e.g., random-access memory, read-only memory, flash memory), electronic storage units (e.g., hard disk), communication interfaces (e.g., network adapter) for communicating with one or more other systems, and peripheral devices, such as cache, other memory, data storage and/or electronic display adapters. In some cases, the communication interface may allow the computer to be in communication with another device such as the autonomous robotic system. The computer may be able to receive input data from the coupled devices such as the autonomous robotic system or a user device for analysis. The memory, storage unit, interface and peripheral devices are in communication with the CPU through a communication bus (solid lines), such as a motherboard. The storage unit can be a data storage unit (or data repository) for storing data.

The CPU can execute a sequence of machine-readable instructions, which can be embodied in a program or software. The instructions may be stored in a memory location. The instructions can be directed to the CPU, which can subsequently program or otherwise configure the CPU to implement methods of the present disclosure. Examples of operations performed by the CPU can include fetch, decode, execute, and write back.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store one or more algorithms and parameters of the robotic system. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers, slate or tablet PC's, smart phones, personal digital assistants, and so on. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in software. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display for providing, for example, images captured by the imaging device. The display may also be capable to provide a user interface. Examples of UI's include, without limitation, a graphical user interface (GUI) and web-based user interface. The UI and GUI can be the same as those described elsewhere herein.

Methods and systems of the present disclosure can be implemented by way of one or more algorithms. An algorithm can be implemented by way of software upon execution by the central processing unit. The algorithms may include, for example, stitch prediction algorithm, location tracking algorithm, tool position correction algorithm and various other methods as described herein.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing one or more aspects of the present disclosure. It is therefore contemplated that the present disclosure shall also cover any such alternatives, modifications, variations or equivalents. It is intended that the following claims define the scope of the present disclosure and that the methods and structures within the scope of these claims and their equivalents be covered thereby.

This listing of claims will replace all prior versions and listings of claims in this application. Applicant reserves the right to pursue any subject matter of any canceled claims in this or any other appropriate patent application. Support for these claims is provided in the remarks following the listing of claims. Listing of the claims

1.-50. (canceled)

51. A method for enabling autonomous or semi-autonomous surgical operations, the method comprising:
(a) capturing an image data stream comprising one or more images of a surgical site;
(b) generating a parametric model for a tissue surface identified in the one or more images;
(c) determining a direction for aligning a tool based in part on the parametric model;
(d) generating an optimal path for automatically moving the tool to perform a surgical procedure at the surgical site; and
(e) generating one or more control signals for controlling i) a movement of the tool based on the optimal path and ii) a tension force applied to the tissue by the tool during the surgical procedure.

52. The method of claim 51, wherein the image data stream is captured using a stereoscopic camera, and wherein the stereoscopic camera is attachable to a joint mechanism that is configured to permit the stereoscopic camera to move in at least three degrees of freedom.

53. The method of claim 52, further comprising before preforming (a), calibrating the stereoscopic camera and determining a registration between the stereoscopic camera and a surgical robot to which the tool is mounted.

54. The method of claim 53, wherein determining the registration comprises calculating a transformation between (i) camera set of spatial coordinates of the stereoscopic camera and (ii) a set of spatial coordinates of the joint mechanism of the surgical robot.

55. The method of claim 51, wherein the one or more images do not contain an image of any portion of the tool.

56. The method of claim 55, further comprising calculating a posture and position of the tool relative to the tissue surface in (c) based at least in part on a registration between a stereoscopic camera and a surgical robot to which the stereoscopic camera is attached.

57. The method of claim 51, wherein the path is a stitching pattern and the tool is a stitching needle, and wherein the stitching pattern is generated based on an opening at the surgical site identified from the one or more images.

58. The method of claim 57, wherein the stitching pattern is generated by identifying a longitudinal axis of the opening and a plurality of anchoring points, and wherein one or more of the plurality anchoring points are determined based in part on a user input.

59. The method of claim 57, wherein the stitching pattern is generated based on a closure changing of an opening at the surgical site during a suturing procedure.

60. The method of claim 51, wherein controlling the tension force in (e) is based on a tension measured in a thread or a usage of the thread during the surgical procedure.

61. The method of claim 51, wherein the tension force is controlled based on a tension or deformation model of a tissue underlying the tissue surface.

62. The method of claim 61, wherein the tension or deformation model of the tissue is constructed based on the parametric model of the tissue surface.

63. The method of claim 61, wherein the tension or deformation model is based at least in part on measurements from an optical sensor.

64. The method of claim 51, wherein the tool is inserted into a body of a subject via a trocar, and wherein the method further comprises compensating a location of the tool by identifying an offset caused by an external force applied to the tool via the trocar.

65. The method of claim 64 wherein the offset is determined by comparing a measured 3D coordinates of the tool with a predicted 3D coordinates of the tool.

66. The method of claim 51, further comprising determining the optimal path based in part on a cyclic movement of one or more features on the surgical site, and wherein the cyclic movement is tracked using the image data stream.

67. The method of claim 51, further comprising tracking a position or an orientation of the tool or a robotic arm controlling the tool using one or more position sensors, and wherein the one or more positions sensors comprise an inertial measurement unit (IMU), an accelerometer, a gyroscope, or a magnetometer.

68. The method of claim 51, wherein the one or more images are captured using a time-of-flight sensor, an RGB-D sensor, or a depth sensor.

69. The method of claim 51, wherein the one or more images comprise a 2D image of the surgical site and a corresponding depth image of the surgical site.

70. The method of claim 51, further comprising processing the one or more images to form a geometric surface model of the surgical site.
